(19) United States
(12) Patent Application Publication
(10) Pub. No.: US 2020/0258030 A1
(43) Pub. Date: Aug. 13, 2020

(54) HEALTH INFORMATION TECHNOLOGY (IT) PROJECT AND CANDIDATE MATCHING SYSTEM AND METHOD

(71) Applicant: Robecca Quammen, Monroe, GA (US)

(72) Inventor: Robecca Quammen, Monroe, GA (US)

(21) Appl. No.: 16/419,063

(22) Filed: May 22, 2019

Related U.S. Application Data
(60) Provisional application No. 62/803,862, filed on Feb. 11, 2019.

(51) Int. Cl. G06Q 10/06; G16H 40/20

(52) U.S. Cl. CPC G06Q 10/063118; G16H 40/20; G06Q 10/06313

(57) ABSTRACT

A system and method for matching projects and candidates in the complex field of healthcare information technology is presented herein. In particular, using a hierarchal selection module that presents a progressive series of categories for selection, a project profile is created. Using the same hierarchal selection module or the same progressive series of categories for selection, a candidate profile is created. The profiles are maintained in a matching management system which will match a project profile with a candidate profile when all of the criteria match in order to provide a highly accurate connection in a highly complex field.

### CLAIM OF PRIORITY/CROSS-REFERENCE TO RELATED APPLICATION

The present application is based on and a claim of priority is made under 35 U.S.C. § 119(e) to provisional patent application Ser. No. 62/803,862, filed on Feb. 11, 2019, the content of which is incorporated herein in its entirety by reference.

### FIELD OF THE INVENTION

The present invention is directed to a system and method for intelligently creating and matching profiles for projects and candidates in the complex field of healthcare information technology using a progressive series of categories that include vendors, technologies, and specific products. This goes beyond general job skills and matches based on precise experiences with various vendor and technology solutions.

### BACKGROUND OF THE INVENTION

The healthcare industry represents a significant amount (nearly 20%) of the gross domestic product (GDP) in the United States. It is a complex industry that requires diverse talent and expertise in all areas of clinical delivery, operations, retail, development, and administration.

Currently, some systems use keyword matching, using resumes, conversations, LinkedIn profiles, etc. in order to attempt to match or connect the workforce to organizations or jobs with very little success, particularly in complex fields including, but not limited to healthcare information technology (IT).

As an example, the healthcare industry can include the use of: robotics performing surgery, radiology imaging, laboratory diagnostic test results and blood matching, medication pumps administering medications to a patient, telemetry monitors, baby abduction prevention, surgical instrument cleaning, diagnostic procedures such as endoscopy and sleep study, etc. In addition, a single healthcare organization can have up to 300 or 500 servers supporting hundreds of discrete software applications as well as complex infrastructure, networking and communication requirements. Furthermore, IT software upgrades occur on a regular basis, making it impossible for long term employees in a traditional healthcare IT shop to keep up and to have firsthand knowledge of the outcome of their work. This gives rise to the expert independent consultants who have increasingly become highly specialized in products and technologies and are in high demand.

IT talent is highly specialized across all third-party software applications, technologies, security, clinical domains, administrative areas, and financial domains. For this reason, many of the current job application boards known today, recruiters, niche firms, and traditional HR departments are not equipped to review, analyze and vet the required skills needed for IT departments and specific projects.

There is, thus, a need in the art for an intelligent information gathering and matching system that can accurately and precisely connect candidates with specific IT experiences and projects that require specific talent. For instance, a growing independent healthcare IT workforce, that is not easily found or marketed, combined with the need for varied and precise expertise in the complex healthcare information technology industry is driving the need for solutions that aid in sourcing talent when and where needed. Independent talent can market specific skills and expertise with the presently proposed solution as opposed to the random selection of key word searches commonly used in commercial job boards.

### SUMMARY OF THE INVENTION

Accordingly, the present invention is directed to a system and method that can be used in the healthcare IT industry, although it is not limited to such. Unlike current keyword matching and resume-based systems which can produce varied and frequently erroneous results, the present invention matches on specific selections made by both a candidate (e.g., an individual with healthcare candidacy or experiences) and a healthcare organization or other entity seeking long- or short-term assistance on a project or a series of projects. The system and method of at least one embodiment can include more than 100,000 combinations of generated profiles or selections based on healthcare venues, healthcare software and technology products, and healthcare information technology roles to allow for the most precise matching possible.

For instance, using the system and method of the present invention, independent healthcare resources or candidates can precisely define skills and expertise across more than 100,000 combinations. The present invention provides healthcare organizations (e.g., recruiters, providers, delivery systems, technology vendors, retail companies, etc.) with a simple, straightforward and logical method to identify specific talent for specific short- and long-term needs.

In at least one exemplary embodiment, the present invention provides four broad categories representative of highly in-demand healthcare IT services: Staff Augmentation, Advisory Services, Interim Leadership, and Outsourcing. Each of these service categories is further developed to represent service delivery venues, topical areas of need within each venue, vendor products and technologies supporting those areas and the specific IT functional, operational or technical tole requirement with requisite experience/skills.

In particular, a candidate seeker, such as a healthcare organization seeking long- or short-term assistance on a project or a series of projects clicks through a series of choices to specifically define talent that is needed. The candidate seeker will then register on the site, if not already completed, and post the project for bidding by indicating the project budget, a narrative description of the project with a descriptive headline, the project starts and due dates, the bidding start and end dates, and the number of consultants needed. Once posted, the system and method will algorithmically and automatically match the project profile with a candidate profile that meets all of the requirements or selections in the project. The organization and the candidate are notified that a match has occurred, and they can choose to begin an online dialogue if desired.

Similarly, the candidate or consultant will register at the site and click through a series of categories to define a candidate profile using the same hierarchal selection module or model of categories as provided to the healthcare organization. Once the profile is finished and submitted, the system and method will notify the candidate if and when any matches are made.

These and other objects, features and advantages of the present invention will become more apparent when the drawings as well as the detailed description are taken into consideration.

### BRIEF DESCRIPTION OF THE DRAWINGS

FIG. 1 is a schematic representation of the system as disclosed in accordance with at least one embodiment of the present invention.

FIG. 2 is a block diagram of the matching management system as disclosed in accordance with at least one embodiment of the present invention.

FIG. 3 is a high-level flow chart illustrating the method as disclosed in accordance with at least one embodiment of the present invention.

FIG. 4A is an exemplary screenshot illustrating a portion of the hierarchal selection module as disclosed in accordance with at least one embodiment the present invention.

FIG. 4B is an exemplary screenshot illustrating another portion (continued from FIG. 4A) of the hierarchal selection module as disclosed in accordance with at least one embodiment the present invention.

FIG. 5A is an exemplary screenshot illustrating project details and project profiles as disclosed in accordance with at least one embodiment herein.

FIG. 5B is another exemplary screenshot illustrating different project details and project profiles as disclosed in accordance with at least one embodiment herein.

FIG. 5C is a yet another exemplary screenshot illustrating different project details and project profiles as disclosed in accordance with at least one embodiment herein.

FIG. 6 is an exemplary screenshot illustrating a candidate or consultant profile as disclosed in accordance with at least one embodiment herein.

Like reference numerals refer to like parts throughout the several views of the drawings provided herein.

### DETAILED DESCRIPTION OF THE INVENTION

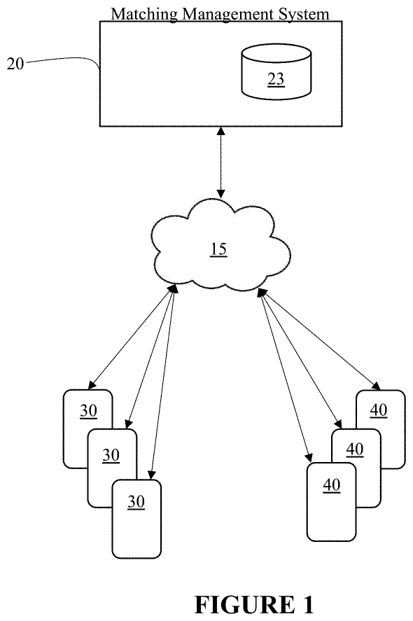

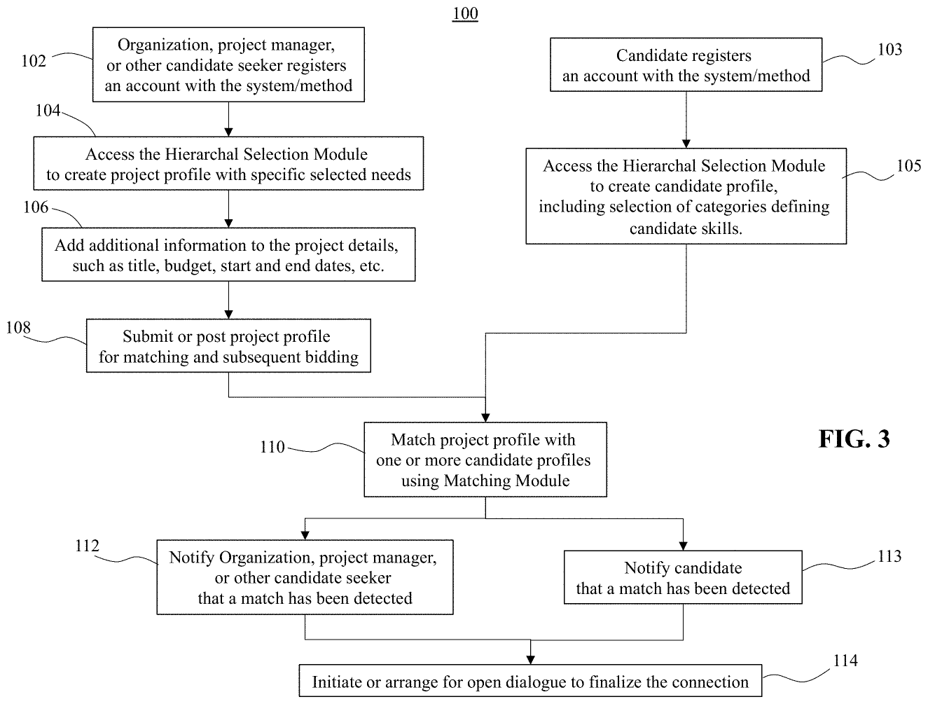

As shown in the accompanying drawings, and with particular reference to FIGS. 1 and 3, the present invention is directed to a system 10 and method 100 for matching a project or a candidate seeker with a project candidate. Specifically, while some embodiments disclosed herein pertain to the health information technology (IT) industry, in that the projects may be related to health IT and/or the one or more candidates may be trained or skilled in the health IT field, some embodiments of the present invention may pertain to other fields either related or completely unrelated to health or IT.

In any event, at least one embodiment of the present invention includes a matching management system, generally referenced as 20 disposed in a communicative relation with at least one computer or communication network 15, such as the World Wide Web, Internet, Intranet, Wide Area Network(s), Telecommunication Network(s) (e.g., 3G, 4G, LTE), etc. In addition, as will be described herein, one or more candidate seekers or user devices operated by candidate seekers, generally referenced as 30, and one or more candidates or user devices operated by candidates, generally referenced as 40, are also communicatively interconnected to the network 15. In this manner, the various users 30, 40 may have controlled access to the matching management system 20 and one or more of the modules thereof, as described herein.

Accordingly, the users 30, 40 may access the matching management system 20 (and in particular, the hierarchal selection module thereof) by visiting a certain website (e.g., via a web browser), opening or executing an application (e.g., a mobile or desktop application), etc., via user or computer device. As should be apparent, and as mentioned above, the computing device(s) accessed or used by the various uses to access the matching management system 20 and/or various features and components of the present invention may include virtually any computer-based system, such as, but not limited to, a desktop computer, laptop computer, mobile computer, tablet, phone, smartphone, media device, game device, personal digital assistant (PDA), etc.

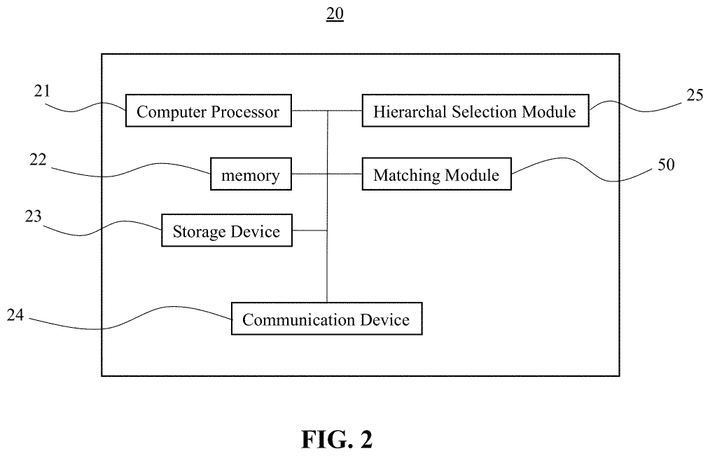

Furthermore, referring now to the schematic representation of the matching management platform/system 20 as represented in FIG. 2, it is noted that the matching management system 20 of at least one embodiment includes, among other components and devices structured to facilitate implementation of the present invention in the intended manner, a computer processor 21, memory 22, a data storage device 23, and a communication or network device(s) 24.

Specifically, as used herein, the computer processor 21 of at least one embodiment may include any device cooperatively structured to execute or implement computer instructions, software, etc., including, for example, the various features and components as described in accordance with at least one embodiment of the present invention and configured to implement the method 100 herein.

Further, the memory device 22 as used herein, may include but is not limited to random access memory (RAM) or other like devices configured to implement the present invention in the intended manner, for example, by at least temporary storing and assisting with the execution of one or more applications, modules, or components capable of implementing the system 10 and method 100 described herein.

The data storage device 23, as used herein, may include a hard disk drive, CD/DVD, USB drive, solid state drive, virtual drive, could-based storage drive, or other types of volatile or non-volatile memory. Moreover, the communication device 24 may include a network communication hardware/software component structured to facilitate communication between the matching management system 20 and the various user devices 30, 40 of the present invention. Accordingly, examples of the matching management system 20 of the present invention may include a web or cloud-based computer or server, desktop computer, laptop computer, or mainframe, although it is contemplated that more mobile devices such as a tablet, smartphone, mobile or handheld computer, etc. can be used in certain implementations.

With reference to the flow chart represented in FIG. 3, the method 100 may begin with the registration of a user, for example, registration of a candidate seeker, as represented at 102 and/or registration of a candidate, as represented at 103. Specifically, the one or more users 30, 40 may, in some implementations, begin by visiting a webpage, web site, or mobile/desktop application where a user account can be created. In the case of the candidate seeker, the account may be by or behalf of an organization, company, business, project manager, individual or other entity who may be seeking assistance, either long or short term, from an outside source. The candidate is the outside source who is seeking a project that matches his or her skill set of which he or she will be able to participate or contribute. Often registration of the account, whether by the candidate or candidate seeker, will include providing basic identifying information, such as a name, physical address, email address, phone number, availability, etc.

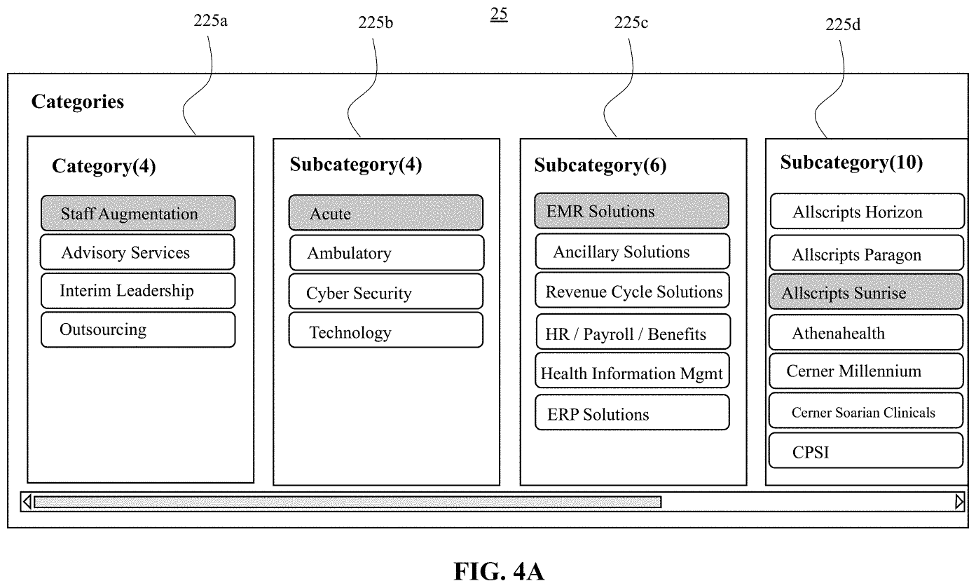

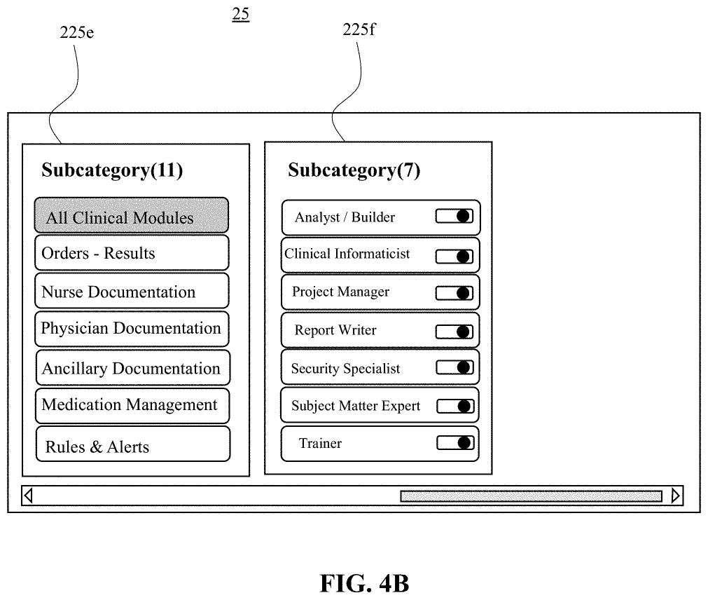

Furthermore, and still referring to the flow chart of FIG. 3, the method 100 includes separately creating a project profile, as shown at 104, and a candidate profile, at 105, using a proprietary and novel hierarchal selection module, as shown at 25 in FIG. 2 and as exemplified in FIGS. 4A and 4B. Specifically, using the hierarchal selection module 25, the candidate seeker 30 will create a project profile 32. Separately, a candidate 40 will create a candidate profile 42 using the hierarchal selection module 25.

Particularly, with reference to exemplary FIGS. 4A and 4B, the hierarchal selection module 25 is a method, process, or procedure implemented in software and/or hardware that intelligently and progressively presents a plurality of categories, subcategories or other selection options 225a-f to the user. In many embodiments, the hierarchal selection module 25 will present the categories 225a-f to the user one at a time, although in some cases more than one category may be presented simultaneously. Particularly, based on the user's selection, the hierarchal selection module 25 will determine which one of the other plurality of categories to present to the user. This determination can be made based upon a selection algorithm that takes into account the user's immediately preceding selection and/or a collection of a plurality of prior selections. Other factors can include information provided when creating the user account to determine what category to present next.

For example, as shown in FIG. 4A, the first category or selection option 225a presented to the user includes a plurality (in the example, four) options from which to select, e.g., “Staff Augmentation,” “Advisory Services,” “Interim Leadership,” and “Outsourcing.” In at least one embodiment, the first category includes a plurality of broad categories that are representative of highly in-demand services, for example, in the healthcare IT industry. Depending on the selection made by the user, the hierarchal selection module 25 will determine which category to present next.

Next, depending on the selection in the first category, a progressive series of categories that include vendors, technologies, and specific products are presented to the user for selection. This goes beyond general job skills and matches based on precise experiences and role-based skills with various vendor and technology solutions.

In the example, “Staff Augmentation” is selected, and based thereupon, the next category/sub-category 225b presented to the user includes a plurality (in the example, four) additional options from which to select, e.g., “Acute,” “Ambulatory,” “Cyber Security,” and “Technology.” In at least one embodiment, if a different selection was made in category 225a, then a different category would be presented instead. Furthermore, in some embodiments, the options presented in 225b are related to the selection made in the previous category 225a, or otherwise further progressively narrow the overall profile. Again, depending on the selection made by the user in 225b, the hierarchal selection module 25 will determine which category to present next.

In the example, “Acute” is selected in category 225b, and based thereupon, the next category/sub-category 225c presented to the user includes a plurality (in the example, six) additional options from which to select, e.g., “EMR Solutions,” “Ancillary Solutions,” “Revenue Cycle Solutions,” “HR/Payroll/Benefits,” “Health Information Management,” and “ERP Solutions.” In at least one embodiment, if a different selection was made in category 225b, then a different category would be presented instead. Furthermore, in some embodiments, these options are related to the selection made in one or more of the previous categories, e.g., 225a or 225b, or otherwise further progressively narrow the overall profile. Again, depending on the selection made by the user, the hierarchal selection module 25 will determine which category to present next.

In the example, “EMR Solutions” is selected in category 225c, and based thereupon, the next category/sub-category 225d presented to the user includes a plurality (in the example, seven) additional options from which to select. In this example, the additional options include a list of products and/or vendor names, e.g., “Allscripts Horizon,” “Allscripts Paragon,” “Allscripts Sunrise,” “Athenahealth,” “Cerner Millennium,” “Cerner Soarian Clinicals,” and “CPSI.” It should be noted that all product names, trademarks and registered trademarks are the property of their respective owners, and use of those names herein does not imply an endorsement or connection therewith. In at least one embodiment, if a different selection was made in category 225c, then a different category would be presented instead. Furthermore, in some embodiments, these options are related to the selection made in one or more of the previous categories, e.g., 225a-225c, or otherwise further progressively narrow the overall profile. Again, depending on the selection made by the user, the hierarchal selection module 25 will determine which category to present next.

It should also be noted that, in this example, specific platforms, products and/or other information technology (IT) product is presented and selected. This allows the system 10 and method 100 of at least one embodiment to match candidates with very specific skills with projects that require candidates with very specific skills. In some cases, the category may also include a list of specific vendors with which the candidate has worked in the past. This allows the system 10 and method 100 of at least one embodiment to match candidates with specific experiences or connections to projects that require candidates to have those experiences or connections.

Next, in the example provided, “Allscripts Sunrise” is selected in category 225d, and based thereupon, the next category/sub-category 225e (shown in FIG. 4B) presented to the user includes a plurality (in the example, seven) additional options from which to select, e.g., “All Clinical Modules,” “Orders—Results,” “Nurse Documentation,” “Physician Documentation,” “Ancillary Documentation,” “Medication Management,” and “Rules & Alerts.” In at least one embodiment, if a different selection was made in category 225d, then a different category would be presented instead. Furthermore, in some embodiments, these options are related to the selection made in one or more of the previous categories, e.g., 225a- 225d, or otherwise further progressively narrow the overall profile. Again, depending on the selection made by the user, the hierarchal selection module 25 will determine which category to present next.

The next, and in this example, final category includes a list of roles from which the user can select. In the embodiment provided, the user can select one or more of the provided roles. In the example, there are seven roles presented, e.g., “Analyst/Builder,” “Clinical Informaticist,” “Project Manager,” “Report Writer,” “Security Specialist,” “Subject Matter Expert,” and “Trainer.” In at least one embodiment, these options are generated or presented to the user by the hierarchal selection module 25 based upon the selections made in any one or more of the previous categories. In the example provided, all of the roles are selected indicating varied skills and experiences.

It should be noted that in at least one embodiment of the present invention, the final or last category the the hierarchal selection module 25 is a role category providing a series of roles that are related to the various previous selections made. The candidate seeker 30 and/or candidate 40 may select one or more roles based upon their work experiences, interests or desires.

Referring again to the flow chart of FIG. 3, once the project profile is created, in at least one embodiment, the candidate seeker 30 will be able to define some additional details referenced as 106. For example, as shown in exemplary screenshots 5A, 5B and 5C, the additional details 35 may include, but certainly are not limited to a title or headline, experience required, project start and end dates, bidding start and end dates, a budget, a brief description, etc. Once all of the details are provided for the project, the candidate seeker 30 is able to submit the project for bidding or matching, as referenced at 108.

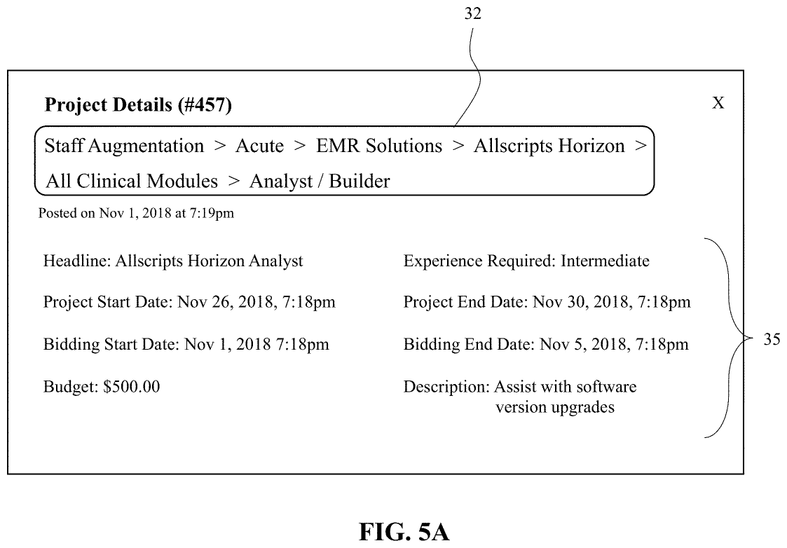

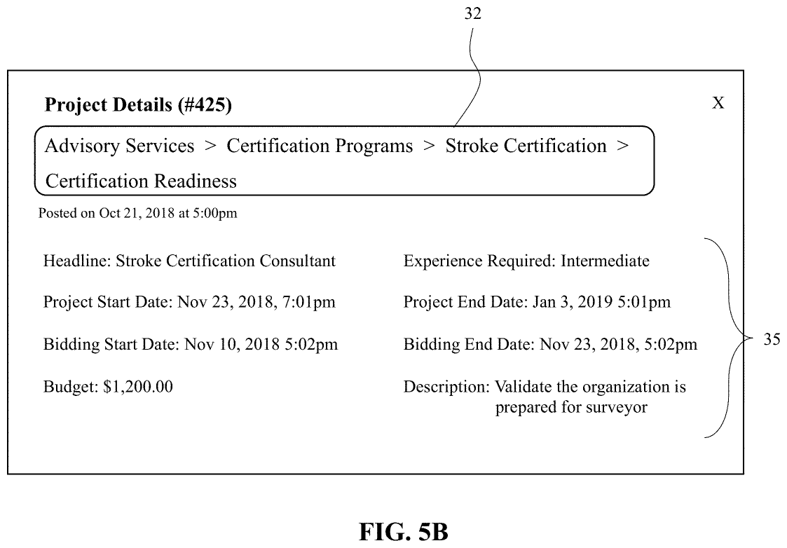

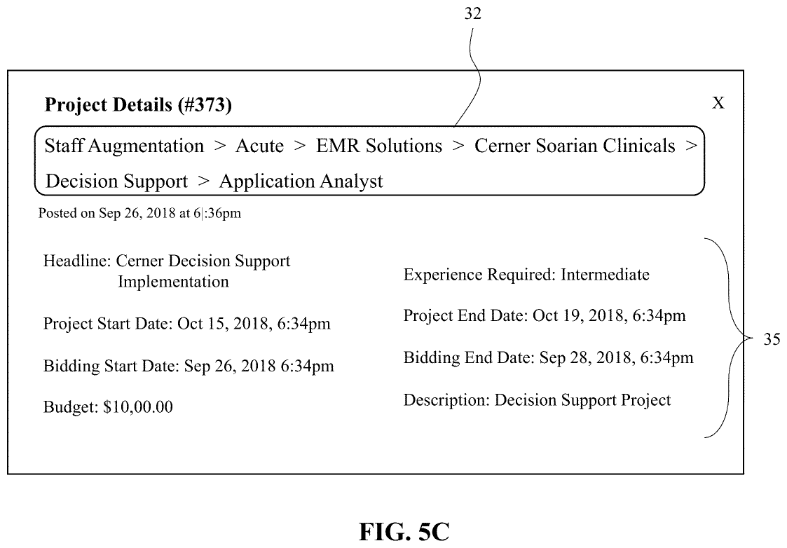

Particularly, FIGS. 5A, 5B and 5C represent three different exemplary screenshots illustrating the project details for three different projects. Within the project details is an organized list of criteria corresponding to the series of selections made by the candidate seeker 30 via the hierarchal selection module 25. Specifically, in the first example of FigureSA, the project profile 32 includes the following organized list or selection criteria: “Staff Augmentation,” “Acute,” “EMR Solutions,” “Cerner Soarian Clinicals,” “Decision Support,” and Role (“Application Analyst.”)

As provided in the other exemplary screenshots of FIGS. 5B and 5C, different project profiles 32 are generated based upon a different set of selections made during or through the hierarchal selection module 25. Furthermore, the additional details 35 is also provided for each of the different projects.

It should be noted that in at least one embodiment, the candidate(s) 40 will not be able to have access to the one or more project details or project profiles 32 generated via the system 10 and/or method 100 of the present invention. In other words, in at least one embodiment, there is no job board or bulletin board that posts the various project profiles 32 generated by the system 10 and method 100. Rather, the system 10 and/or method 100 of at least one embodiment presented herein will retain the project details and/or project profiles 32 and attempt to match them with submitted candidate profiles 42.

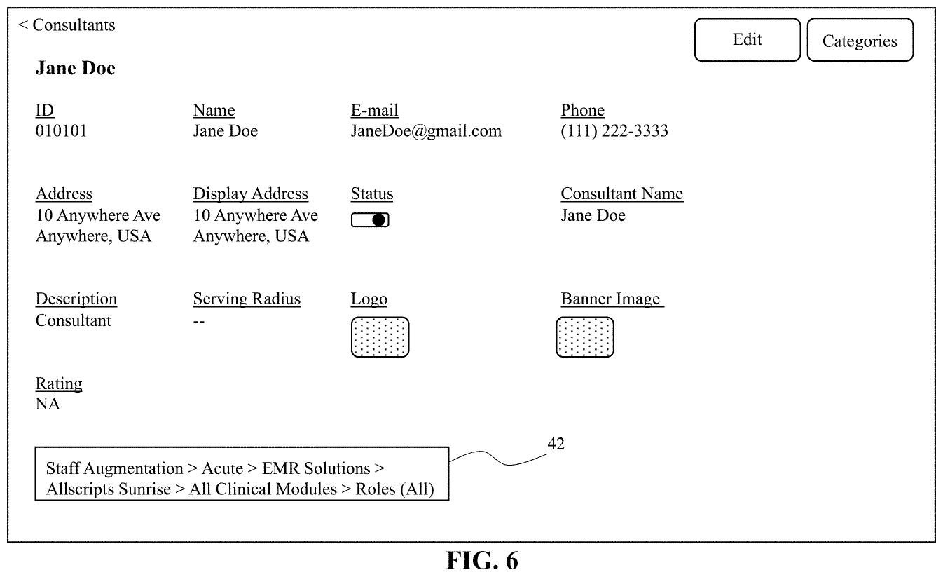

For instance, FIG. 6 provides an exemplary candidate account with a candidate profile 42 created or generated via the hierarchal selection module 25. In this example, the candidate profile includes the organized list of selections made by the candidate 40 using the hierarchal selection module 25.

It should be noted that in at least one embodiment, the same hierarchal selection module 25 is used to create or generate the project profile 32 and the candidate profile 42. In other words, both the candidate seeker 30 and the candidate 40 will be presented with the same hierarchal selection module 25 or the same plurality of progressive categories providing an opportunity for both the project profile 32 and the candidate profile 42 created thereby to be identical.

Furthermore, referring again to the flow chart of FIG. 3, as provided at 110, the method 100 includes matching a project profile 32 with a candidate profile 42 via a matching module 50 (shown in FIG. 2) based upon the selections provided by the candidate seeker 30 and candidate 40, respectively. For example, in one embodiment, in order for a project profile 32 to “match” a candidate profile 42, the project profile 32 and the candidate profile 42 must match identically or otherwise each of the selections in each of the categories must match. In other words, in order for a candidate profile to match the project profile presented in exemplary screenshot of FIG. 5A, the candidate profile must include an organized list of selections that match completely or exactly with each of the organized list of selections of the project profile. In this manner, in order for the candidate profile to match the project profile of at least one embodiment, the selections submitted during the questionnaire or selection module for the candidate profile and the project profile must be a one-to-one or exact match. For example, a matching candidate profile to the project profile 32 of FIG. 5A, must include “Staff Augmentation,” “Acute,” “EMR Solutions,” “Cerner Soarian Clinicals,” “Decision Support,” and “Application Analyst.”

To be clear, in the role category, which is often the last category, or in other categories that may allow for more than one selection to be made, there must be some overlap for there to be an identical match. For example, if the project profile includes “Analyst/Builder,” and the candidate profile includes “Analyst/Builder” and “Project Manager,” then there is overlap in the selections and an identical match is made.

It should also be noted that in some embodiments, a match can be determined even if there is not an identical match, so long as a certain predetermined percentage or predetermined amount of selections between the two profiles match.

In any event, when the system 10 and/or method 100 determines that a match has been made, as shown at 112 and 113, the system 10 and/or method 100 can notify the candidate seeker 30 and the candidate 40, for example, by email, phone, text message, SMS or MMS message, push notification, etc. This allows the matched parties to open a direct communication dialogue, as represented at 114, via email, phone, text message, SMS or MMS messaging, or in person, if desired.

With the specific matching algorithm and novel hierarchal selection module 25 used and disclosed in connection with the present invention, matches between candidate and candidate seekers will have a high degree of success and compatibility.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention. This written description provides an illustrative explanation and/or account of the present invention. It may be possible to deliver equivalent benefits using variations of the specific embodiments, without departing from the inventive concept. This description and these drawings, therefore, are to be regarded as illustrative and not restrictive.

Now that the invention has been described,

What is claimed is:

1. A project and candidate matching method comprising:
creating, via a matching management system, a project profile based upon a series of selections made by a candidate seeker via a hierarchical selection module, the project profile comprising an organized list of criteria corresponding to the series of selections made by the candidate seeker via the hierarchal selection module, the matching management system comprising at least one computer processor, a memory and a storage device,
creating, via the matching management system, a candidate profile based upon a series of selections made by a candidate via the hierarchal selection module, the candidate profile comprising an organized list of criteria corresponding to the series of selections made by the candidate via the hierarchal selection module, and
matching, via the matching management system, the project profile and the candidate profile wherein the organized list of criteria of the project profile and the organized list of the candidate profile comprise a match of common selections.

2. The method as recited in claim 1 further comprising presenting, via the hierarchal selection module, to the candidate seeker, a plurality of progressive categories, wherein each of the plurality of progressive categories presented to the candidate seeker comprises a plurality of possible selections.

3. The method as recited in claim 2 wherein a selection made by the candidate seeker on one of the plurality of progressive categories presented to the candidate seeker determines which one of the plurality of progressive categories is presented to the candidate seeker next.

4. The method as recited in claim 3 further comprising presenting, via the hierarchal selection module, to the candidate, a plurality of progressive categories, wherein each of the plurality of progressive categories presented to the candidate comprises a plurality of possible selections.

5. The method as recited in claim 4 wherein a selection made by the candidate on one of the plurality of progressive categories presented to the candidate determines which one of the plurality of progressive categories is presented to the candidate next.

6. The method as recited in claim 5 wherein the project profile and the candidate profile are created using the same hierarchal selection module with the same plurality of progressive categories.

7. The method as recited in claim 5 wherein the project profile and the candidate profile are created using the same plurality of progressive categories.

8. The method as recited in claim 5 further comprising matching, via the matching management system, the project profile and the candidate profile wherein the organized list of criteria of the project profile and the organized list of the candidate profile comprise identical selections.

9. The method as recited in claim 5 wherein the project profile is not accessible by a candidate via the matching management system.

10. The method as recited in claim 5 wherein at least one of the plurality of progressive categories comprise an identification of a specific information technology product.

11. The method as recited in claim 5 wherein at least one of the plurality of progressive categories comprise an identification of a specific information technology vendor.

12. The method as recited in claim 5 wherein the project profile comprises a healthcare information technology project and wherein the candidate comprises a healthcare information technology candidate.

13. A healthcare information technology (IT) project and candidate matching method comprising:
creating, via a matching management system, a project profile based upon a series of selections made by a candidate seeker via a hierarchical selection module, the project profile comprising an organized list of criteria corresponding to the series of selections made by the candidate seeker via the hierarchal selection module, the matching management system comprising at least one computer processor, a memory and a storage device,
wherein creating the project profile comprises:
presenting, via the hierarchal selection module, to the candidate seeker, a plurality of progressive categories, wherein each of the plurality of progressive categories presented to the candidate seeker comprises a plurality of possible selections related to the healthcare information technology field,
wherein a selection made by the candidate seeker on one of the plurality of progressive categories presented to the candidate seeker determines which one of the plurality of progressive categories is presented to the candidate seeker next, and
compiling each of the selections made by the candidate seeker into the organized list of criteria creating the project profile,
defining the project profile as corresponding to a healthcare information technology project,
creating, via the matching management system, a candidate profile based upon a series of selections made by a candidate via the hierarchal selection module, the candidate profile comprising an organized list of criteria corresponding to the series of selections made by the candidate via the hierarchal selection module,
wherein creating the candidate profile comprises:
presenting, via the hierarchal selection module, to the candidate, a plurality of progressive categories, wherein each of the plurality of progressive categories presented to the candidate comprises a plurality of possible selections related to the healthcare information technology field,
wherein a selection made by the candidate on one of the plurality of progressive categories presented to the candidate determines which one of the plurality of progressive categories is presented to the candidate next, and
compiling each of the selections made by the candidate into the organized list of criteria creating the candidate profile,
defining the candidate profile as corresponding to a healthcare information technology candidate,
matching, via the matching management system, the project profile and the candidate profile wherein the organized list of criteria of the project profile and the organized list of criteria of the candidate profile comprise identical selections.

14. The method as recited in claim 13 wherein the project profile and the candidate profile are created using the same plurality of progressive categories.

15. The method as recited in claim 14 wherein at least one of the plurality of progressive categories comprise an identification of a specific information technology product.

16. The method as recited in claim 14 wherein at least one of the plurality of progressive categories comprise an identification of a specific information technology vendor.

17. A healthcare information technology (IT) project and candidate matching system comprising:
a matching management system disposed in a communicative relation with a network, the matching management system comprising at least one computer processor, a memory and a storage device,
said matching management system comprising a hierarchal selection module, the hierarchal selection module being configured to create a project profile and a candidate profile,
wherein said hierarchal selection module is configured present a plurality of progressive categories to a candidate seeker via the network, wherein each of the plurality of progressive categories presented to the candidate seeker comprises a plurality of possible selections related to the healthcare information technology field,
wherein a selection made by the candidate seeker on one of the plurality of progressive categories presented to the candidate seeker determines which one of the plurality of progressive categories is presented to the candidate seeker next by the hierarchal selection module, and
wherein said hierarchal selection module is configured to compile each of the selections made by the candidate seeker into an organized list of criteria to create the project profile,
wherein said hierarchal selection module is configured present a plurality of progressive categories to a candidate via the network, wherein each of the plurality of progressive categories presented to the candidate comprises a plurality of possible selections related to the healthcare information technology field,
wherein a selection made by the candidate on one of the plurality of progressive categories presented to the candidate determines which one of the plurality of progressive categories is presented to the candidate next by the hierarchal selection module, and
wherein said hierarchal selection module is configured to compile each of the selections made by the candidate into an organized list of criteria to create the candidate profile,
said matching management system further comprising a matching module configured to analyze said project profile and said candidate profile, wherein said project profile and said candidate profile are considered matched by the matching module when the organized list of criteria of the project profile and the organized list of criteria of the candidate profile comprise identical selections.
